(19) United States
(12) Patent Application Publication
(10) Pub. No.: US 2011/0224397 A1
(43) Pub. Date: Sep. 15, 2011

(54) PROCESS FOR PREPARING POLYOLS USING BASE CATALYSIS

(75) Inventors: Thomas OSTROWSKI, Mannheim (DE); Anne-Kathrin Merten, Lauchhammer (DE); Achim Löffler, Speyer (DE); Hermann Graf, Mutterstadt (DE)

(73) Assignee: BASF SE, Ludwigshafen (DE)

(21) Appl. No.: 13/042,781

(22) Filed: Mar. 8, 2011

Related U.S. Application Data
(60) Provisional application No. 61/311,782, filed on Mar. 9, 2010.

(51) Int. Cl. C08G 18/48; C07C 41/26; C07C 43/03

(52) U.S. Cl. 528/76; 568/606

(57) ABSTRACT

The present invention relates to a process for preparing polyetherols in the presence of basic catalysts, wherein propylene oxide (PO) and/or ethylene oxide (EO) whose summated contents
of aldehydes, expressed by the contents determined by gas chromatography or titrimetrically using the bisulfite method and calculated for propionaldehyde, do not exceed the value of 300 ppm and
of allyl alcohol determined by gas chromatography do not exceed the value of 2500 ppm and
of water determined by Karl-Fischer titration do not exceed the value of 1700 ppm and
of acid, expressed by the contents determined by acid-base titration and calculated for acetic acid, do not exceed the value of 100 ppm and
of carbon dioxide determined by KOH titration do not exceed the value of 500 ppm are used.

This patent application claims the benefit of pending U.S. provisional patent application Ser. No. 61/311,782 filed Mar. 9, 2010, incorporated in its entirety herein by reference.

### BACKGROUND

The invention relates to a process for preparing polyether alcohols (polyetherols) by catalyzed addition of alkylene oxides onto solid starter substances, primarily sucrose, and the use of these polyether alcohols for producing polyurethanes (PUR), in particular PUR foams.

The preparation of polyether alcohols by anionic polymerization of alkylene oxides has been known for a long time.

Further details may be found, for example, in Kunststoffhandbuch, volume VII, Polyurethane, Carl-Hanser-Verlag, Munich, 1st edition 1966, edited by Dr. R. Vieweg and Dr. A. Höchtlen, and 2nd edition 1983 and 3rd edition 1993, edited by Dr. G. Oertel, or M. Szycher, Szycher's Handbook of Polyurethanes, CRC Press, New York 1999, chapter 5 “Polyols”.

The addition reaction of the alkylene oxides is usually carried out using catalysts. In industry, predominantly basic, in particular alkaline, catalysts are used for this purpose.

Basic compounds such as alkali metal hydroxides and alkaline earth metal hydroxides are among the standard catalysts in the preparation of polyether alcohols; potassium hydroxide (KOH) is the most widely used.

The preparation of polyether alcohols is described in M. Ionescu, “Chemistry and Technology of Polyols for Polyurethanes”, Rapra Technology, 2005.

Frequently used alkylene oxide starting materials for the preparation of polyether alcohols are propylene oxide (PO) and/or ethylene oxide (EO).

It is a continuing task in industry to improve the quality of the products, to optimize the process and to ensure plant safety.

Malfunctions in the process can occur when the at least one alkylene oxide used, in particular propylene oxide (PO) and/or ethylene oxide (EO) has an excessively high content of by-products (interfering substances).

Impurities can also be introduced into the process by the starting materials, e.g. alcohols; however, the present disclosure is concerned particularly with impurities in the alkylene oxide starting materials such as EO and PO.

For example, aldehydes which can occur in PO both from the epichlorohydrin process and from the SM/PO and MTBE-PO process (SM=styrene monomer; MTBE=methyl tert-butyl ether) can cause malfunctions in a number of ways:
a) a number of aldol condensations can occur in the presence of the basic catalysts and these lead to colored compounds and cause a deterioration in the color number of the polyol,
b) cyclic acetals can be formed with low molecular weight glycols, in particular dipropylene glycol and glycerol, present in the starter mixture and can be removed only with difficulty or not at all in the work-up. Since they bind the hydroxyl groups of the starter molecules, they reduce the functionality of the end product and/or deactivate part of the starter molecules, so that a polyol having the wrong OH number results. As unreactive material, they falsely indicate a lower OH number (the OH number is based on the weight used, “dilution effect”). When the polyol batch is used in polyurethane formulations, an impairment of the quality can occur since the desired ratio of NCO to OH functionality is no longer correct because of the excessively low OH number.
c) the odor attributable to aldehydes and their derivatives can be a problem not only in the synthesis but also in the use of the polyurethane produced from the polyol.

Allyl alcohol, which can be formed by rearrangement of the PO, acts as monofunctional starter and reduces the functionality of the polyol. This results in incorrect values of the OH number and viscosity and also deviations from the correct properties in the polyurethanes produced therefrom.

Water as ubiquitous interfering substance (when present in unknown amounts) acts as bifunctional starter and alters the functionality, with consequences as indicated for allyl alcohol.

Acid can be present as mineral acid (e.g. as hydrochloric acid in PO from the epichlorohydrin process) and/or as organic carboxylic acid (in the case of PO from the SM/PO or MTBE/PO process). Mineral acid deactivates the basic catalysts used in the polyol synthesis, in particular when tertiary amine catalysts are used as cocatalysts, since these are less active compared to the alkali/alkaline earth metal hydroxides.

Carboxylic acids have a similar effect, but additionally as monofunctional starter. The alkoxylated carboxylic acid primarily formed can be hydrolyzed further by the basic catalyst in the presence of traces of water, so that diols are forms with liberation of the carboxylic acid and these lead to an altered functionality. Acetic acid therefore effectively has the same effect as the equivalent amount of water.

Carbon dioxide reacts with the basic catalyst to form inorganic carbonates and partially deactivates the catalyst. When ethylene glycol or 1,2-propylene glycol is used as starter, cyclic carbonates which have the functionality zero are formed by base catalysis. The resulting polyol then does not have the intended functionality and the OH number is too high since the carbon dioxide consumes propylene oxide which is then not available for buildup of the chain.

It was therefore an object of the invention to improve the base-catalyzed process for preparing polyols using at least one alkylene oxide, such as propylene oxide and/or ethylene oxide as starting materials so as to avoid the abovementioned problems; this was directed in particular to the KOH-catalyzed process.

### FIGURES

FIG. 1 shows by way of example the OH number of a polyol as a function of the content of allyl alcohol in PO.

FIG. 2 shows by way of example the viscosity of a polyol as a function of the content of allyl alcohol in PO.

### DESCRIPTION OF THE INVENTION

The problem was solved according to the invention by carrying out alkoxylations using only (at least one) alkylene oxide, in particular PO and/or EO, whose summated content(s) of the following interfering substances does/do not exceed the following defined threshold values:
content of aldehydes, expressed by the contents determined by gas chromatography or titrimetrically using the bisulfite method and calculated for propionaldehyde, of ≦300 ppm, preferably ≦120 ppm, particularly preferably ≦60 ppm
content of allyl alcohol determined by gas chromatography of ≦2500 ppm, preferably ≦1700 ppm, particularly preferably ≦1000 ppm
content of water determined by Karl-Fischer titration of ≦1700 ppm, preferably ≦500 ppm, particularly preferably ≦200 ppm
content of acid in the form of mineral acid and/or organic acid, expressed by the contents determined by acid-base titration and calculated for acetic acid, of ≦100 ppm, preferably ≦40 ppm, particularly preferably ≦20 ppm
content of carbon dioxide determined by KOH titration of ≦500 ppm, preferably ≦200 ppm, particularly preferably ≦100 ppm

Here, all of the abovementioned limits for the summated contents of the interfering substances are adhered to.

This means, for example, that when using only a propylene oxide as alkylene oxide starting compound, both the content of aldehydes in the propylene oxide does not exceed the value of 300 ppm and the content of carbon dioxide in the propylene oxide also does not exceed the value of 500 ppm. On the other hand, if, for example, propylene oxide and ethylene oxide are used as alkylene oxide starting compounds, this means, inter alia, that both the summated content of aldehydes in the propylene oxide and in the ethylene oxide does not exceed the value of 300 ppm and the summated content of allyl alcohol in the propylene oxide and in the ethylene oxide also does not exceed the value of 2500 ppm.

The invention accordingly provides a process for the base-catalyzed preparation of polyetherols, wherein at least one alkylene oxide, in particular propylene oxide (PO) and/or ethylene oxide (EO), whose summated contents
a) of aldehydes, expressed by the contents determined by gas chromatography or titrimetrically using the bisulfite method and calculated for propionaldehyde, do not exceed the value of 300 ppm and
b) of allyl alcohol determined by gas chromatography do not exceed the value of 2500 ppm and
c) of water determined by Karl-Fischer titration do not exceed the value of 1700 ppm and
d) of acid, expressed by the contents determined by acid-base titration and calculated for acetic acid, do not exceed the value of 100 ppm and
e) of carbon dioxide determined by KOH titration do not exceed the value of 500 ppm, is used.

The process of the invention for preparing polyetherols in the presence of basic catalysts is preferably carried out using a propylene oxide (PO) and/or an ethylene oxide (EO) whose summated contents of aldehydes, expressed by the contents determined by gas chromatography or titrimetrically using the bisulfite method and calculated for propionaldehyde, do not exceed the value of 120 ppm.

In a further preferred embodiment of the process of the invention for preparing polyetherols in the presence of basic catalysts, the process is carried out using a propylene oxide (PO) and/or an ethylene oxide (EO) whose summated contents of allyl alcohol determined by gas chromatography do not exceed the value of 1000 ppm.

In a further preferred embodiment of the process of the invention for preparing polyetherols in the presence of basic catalysts, the process is carried out using a propylene oxide (PO) and/or an ethylene oxide (EO) whose summated contents of water determined by Karl-Fischer titration do not exceed the value of 500 ppm, preferably 200 ppm.

In a further preferred embodiment of the process of the invention for preparing polyetherols in the presence of basic catalysts, the process is carried out using propylene oxide (PO) and/or an ethylene oxide (EO) whose summated contents of acid, expressed by the contents determined by acid-base titration and calculated for acetic acid, do not exceed the value of 40 ppm.

In a further preferred embodiment of the process of the invention for preparing polyetherols in the presence of basic catalysts, the process is carried out using a propylene oxide (PO) and/or an ethylene oxide (EO) whose summated contents of carbon dioxide determined by KOH titration do not exceed the value of 200 ppm.

In a further preferred embodiment of the process of the invention for preparing polyetherols in the presence of basic catalysts, the process is carried out using only EO and the content of water determined by Karl-Fischer titration does not exceed the value of 70 ppm, preferably 50 ppm.

The analyses of the respective interfering substances were carried out as follows:
aldehyde content, bisulfite method: Houben-Weyl, Method. d. Org. Chemie vol. 2, Analyt. Method., Thieme Verl. Stuttgart 1953, pp. 463/464 (volumetric analytical method)
aldehyde content (DNPH+HPLC (DNPH=2,4-dinitrophenylhydrazine; HPLC=High Performance Liquid Chromatography)): VDI 3862 part 2; ketones interfere in the bisulfite method. The DNPH method is therefore employed when further impurities in the form of ketones are presumed.
allyl alcohol and carbon dioxide by gas chromatography: analogous to DIN 51405; or carbon dioxide by KOH titration. When further impurities in the form of additional acids are presumed, carbon dioxide is determined by gas chromatography.
acetic acid by acid-base titration: DIN EN 62021-2 (colorimeter.) or DIN (DIN=deutsche Industrienorm [German industrial standard]) 12634 (potentiometr.)
water content by the Karl Fischer method: DIN EN 13267

Possible starters for the process are, for example:
a) monohydric and polyhydric alcohols having a functionality F=1-8, for example MEG (monoethylene glycol), DEG (diethylene glycol), TEG (triethylene glycol), PEG (polyethylene glycol); MPG (monopropylene glycol), DPG (dipropylene glycol), TPG (tripropylene glycol), PPG (polypropylene glycol); PTHF (polytetrahydrofuran), glycerol, glycerol-alkoxylate having a molar mass of <10 000, TMP (trimethylol-propane), TME, (trimethylolethane), NPG (neopentyl glycol), allyl alcohol alkoxylate having a molar mass of <1000, sugars and sugar derivatives such as sucrose or sorbitol, bisphenol A, bisphenol F, pentaerythritol, degraded starch, water, mixtures thereof;
b) monofunctional and polyfunctional amines (aliphatic and aromatic) such as ethylenediamine, triethanolamine or the various isomers of toluenediamine such as 2,4-, 2,6- or vic-TDA,
c) hydroxycarboxylic acids, hydroxyaldehydes, hydroxyketones; tridecanol N and polymers thereof; ester of acrylic acid and methacrylic acid with bifunctional alcohols, e.g. HEA (hydroxyethyl acrylate), HPA (hydroxypropyl acrylate), HEMA (hydroxyethyl methacrylate), HPMA (hydroxypropyl methacrylate), vinyl ethers such as HBVE (hydroxybutyl vinyl ether); isoprenol; polyesterols; lower alkoxylates of the abovementioned starters, in particular of sucrose, sorbitol; polyesterols,
d) vegetable oils having hydroxyl groups or vegetable oils into which hydroxyl groups have been introduced by chemical modification, e.g. soybean oil or castor oil.

The starters can be initially charged at the beginning of the reaction or, if appropriate, also be introduced during the process.

The alkylene oxides used are preferably selected from the group consisting of ethylene oxide and propylene oxide.

The basic catalysts are preferably selected from the group consisting of alkali metal hydroxides and alkaline earth metal hydroxides and monofunctional, bifunctional or trifunctional amines; particular preference is given to potassium hydroxide.

The process can be carried out as a random or block copolymerization using different alkylene oxides. For example, EO/PO mixtures can be randomly polymerized by introducing EO and PO as a mixture or can be polymerized blockwise by firstly introducing pure PO and then introducing pure EO, or vice versa.

For example, the process of the invention can be carried out in a stirred vessel which can be equipped with at least one internal heat exchanger and/or at least one external heat exchanger.

The catalyst can all be added at the beginning of the reaction or the catalyst can be introduced in portions over the reaction time. The starter substance or substances can be added similarly.

The process of the invention can be carried out as a batch, semibatch or continuous process.

The reaction of the starter substance with the alkylene oxides is carried out at the customary pressures in the range from 0.1 to 1.0 MPa and the customary temperatures in the range from 80 to 160° C. The introduction of the alkylene oxides is usually followed by an after-reaction phase to allow the alkylene oxides to react completely. The crude polyether alcohol obtained in this way is freed of unreacted alkylene oxide and volatile compounds by distillation or stripping, preferably under reduced pressure, dewatered and worked up by neutralization with acid and removal of the salts formed.

When amines are used as catalysts, these can remain in the polyol.

The process of the invention for preparing polyetherols in the presence of basic catalysts also encompasses the polyetherols which can be prepared using the process of the invention.

The polyether alcohols which can be prepared by the process of the invention can preferably be reacted with polyisocyanates to form polyurethanes.

The use of the polyether alcohols prepared according to the invention in the production of polyurethanes allows the properties of the end product to be accurately predicted since the polyether alcohols prepared according to the invention have precisely defined and predictable properties because of the absence of undesirable secondary reactions with interfering substances or impurities in the production process. This makes the tailored production of polyurethanes having particular properties possible. In addition, it was for the first time possible to achieve some specifications desired in polyurethanes, e.g. very little color, when using the polyether alcohols prepared according to the invention.

The use of the polyetherols which can be prepared according to the invention for producing PUR (polyurethane) foams is particularly preferred.

### EXAMPLES

The invention is illustrated below with the aid of selected examples. However, the examples do not in any way restrict the scope of the invention; they are to be interpreted purely as illustrative.

### A) Experiment:

5.4 g of glycerol were placed in a 300 ml stainless steel autoclave 1.33 g of 45 percent aqueous potassium hydroxide solution were added, the vessel was closed, heated to 120° C. while stirring and maintained under a reduced pressure of 20 mbar for 1 hour. A nitrogen pressure of 1 bar was subsequently set. 194.6 g of pure propylene oxide were metered in at 110-130° C. via a pressure line over a period of about 5 hours. When the pressure remained constant, the autoclave was cooled, vacuum was briefly applied, the vacuum was broken and 1.25 g of 85 percent phosphoric acid were added at room temperature while stirring. After 30 minutes, vacuum was applied and at the same time a gentle stream of nitrogen was introduced. The batch was subsequently filtered through a plate filter. The OH number and the viscosity was subsequently determined.

OH number: 42.1 mg KOH/g
Viscosity: 670 mPa*s/25° C.

The OH number and the viscosity are determined by standard methods with which a person skilled in the art will be familiar.

The term “pure propylene oxide” refers to a propylene oxide which comprises interfering substances and impurities only up to the following upper limits:
aldehydes (as propionaldehyde)≦60 ppm
allyl alcohol≦1700
water≦500 ppm
acid (as HOAc)≦20 ppm
carbon dioxide≦100 ppm
   B) Experiments with Propionaldehyde Contamination:
   1. Experiment A) was repeated but 0.973 g of propionaldehyde (corresponding to 5000 ppm based on 194.6 g of crude PO) was added after the introduction of nitrogen and 193.6 g of pure PO were introduced.
   OH number: 30.2 mg KOH/g
   Viscosity: 960 mPa*s/25° C.
   2. The experiment was carried out as for batch B1 but using 0.389 g of propionaldehyde (corresponding to 2000 ppm based on 194.6 g of crude PO) and 194.3 g of pure PO. The aldehyde was introduced by means of a syringe through a septum into the reaction vessel.
   OH number: 37.4 mg KOH/g
   Viscosity: 840 mPa*s/25° C.
   3. In further batches, the amount of propionaldehyde was successively reduced. OH number and viscosity were plotted graphically against the amount of propionaldehyde and the value of the amount of propionaldehyde for which OH number and viscosity were identical within the limits of accuracy to those of experiment A determined by extrapolation. This value was approx. 300 ppm.
   C) Experiments with Allyl Alcohol Contamination:
   Experiment A) was repeated but 0.973 g of allyl alcohol (corresponding to 5000 ppm based on 194.6 g of crude PO) was added after the introduction of nitrogen and 193.6 g of pure PO were introduced.

The further experiments were carried out analogously. The results are summarized in table 1:

TABLE 1

| Allyl alcohol | Allyl alcohol | Pure PO | OH number | Viscosity |
|---|---|---|---|---|
| [ppm] | [g] | [g] | [mg KOH/g] | [mPa * s/25° C.] |
| 5000 | 0.973 | 193.6 | 54.2 | 550 |
| 1500 | 0.292 | 194.3 | 46.2 | 610 |
| 1000 | 0.195 | 194.4 | 44.5 | 630 |
| 500 | 0.097 | 194.5 | 43.3 | 645 |
| 0 | 0.000 | 194.6 | 42.1 | 660 |

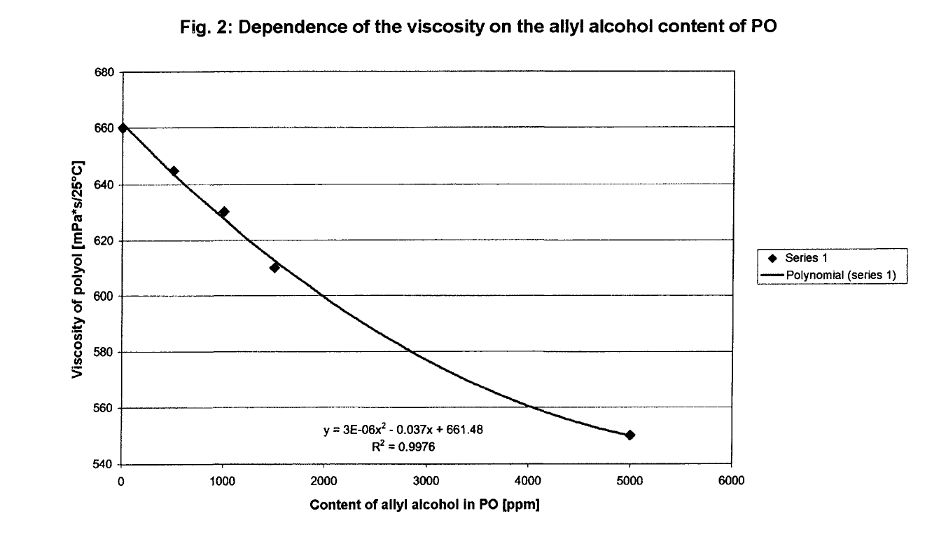

To determine the maximum permissible amount of allyl alcohol contamination, the OH number and the viscosity were plotted against this amount and a polynomial curve was fitted by the method of least squares, as indicated in FIG. 2.

This was used to determine the maximum permissible amount of allyl alcohol based on the requirement that OH number and viscosity should not differ by more than the limits of accuracy from the comparative batch without contamination. The limit of accuracy for the OH number was taken to be one unit (i.e. 1 mg KOH/g), and that for the viscosity was taken to be ±3% of the measured value (rotational viscometer; at 660 mPa*s accordingly about 20 mPa*s). This gave 500 ppm as the maximum amount.

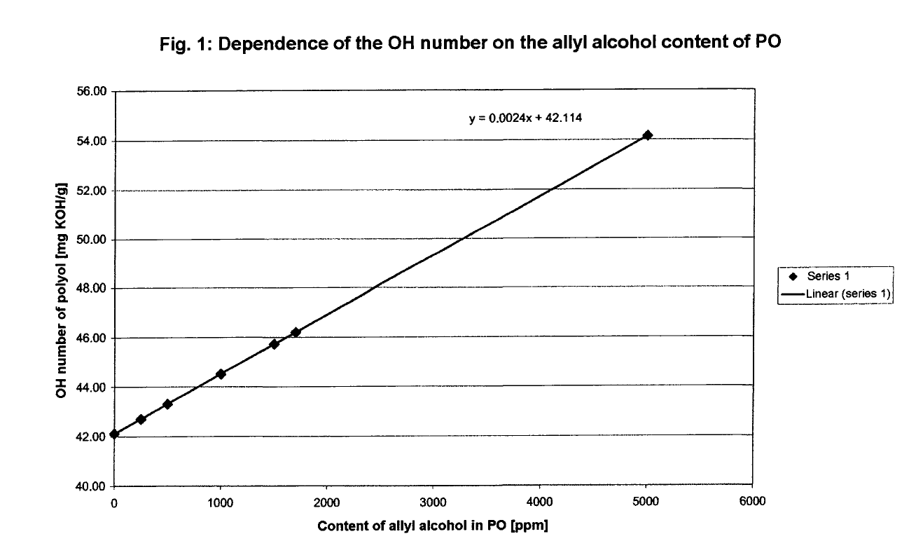

FIG. 1 shows the curve for the OH number; FIG. 2 shows that for the viscosity.

D) Experiments with Water Contamination:

See table 2. The procedure of A) was repeated but the amounts indicated in table 2, column 1, of water were added after the introduction of nitrogen. The PO used comprised 250 ppm of water. The amount of water added was selected so that the total amounts indicated in table 2, column 2, resulted.

TABLE 2

| | | | Amount of PO | | |
|---|---|---|---|---|---|
| Water | Water | Water, | comprising | | Viscosity |
| added | added | total | 250 ppm | OH number | [mPa * s/ |
| [ppm] | [g] | [ppm] | of H₂O [g] | [mg KOH/g] | 25° C.] |
| 4750 | 0.924 | 5000 | 194.6 | 80.9 | 170 |
| 1250 | 0.243 | 1500 | 194.6 | 53.8 | 510 |
| 750 | 0.146 | 1000 | 194.6 | 49.9 | 540 |
| 250 | 0.049 | 500 | 194.6 | 46.0 | 610 |
| 0 | 0.0 | 250 | 194.6 | 44.1 | 640 |

The maximum permissible amount was determined as described in B). The amount was in this way found to be 500 ppm.

However, a person skilled in the art will know that it is precisely in the case of the interfering substance water that decreases in quality of the product which are still acceptable for many applications generally result, even at relatively high contents in the alkylene oxide used.

E) Experiments with Acetic Acid Contamination:

See table 3, procedure as under A), but the amounts of glacial acetic acid indicated in table 3 were added after the introduction of nitrogen.

TABLE 3

| Acetic acid | Acetic acid | Pure PO | OH number | Viscosity |
|---|---|---|---|---|
| [ppm] | [g] | [g] | [mg KOH/g] | [mPa * s/25° C.] |
| 5000 | 0.973 | 193.6 | 53.9 | 510 |
| 1500 | 0.292 | 194.3 | 45.7 | 620 |
| 500 | 0.097 | 194.5 | 43.3 | 645 |
| 250 | 0.049 | 194.6 | 42.70 | 650 |
| 0 | 0.000 | 194.6 | 42.1 | 660 |

The maximum permissible amount was determined as described in B). The amount was in this way found to be 100 ppm.

F) Experiments with Carbon Dioxide Contamination:

See table 4. The experiments were carried out as under A) but the amounts indicated in table 4 of carbon dioxide were introduced by means of a gastight syringe via a septum into the reactor after the introduction of nitrogen.

TABLE 4

| | | | | Viscosity |
|---|---|---|---|---|
| Carbon dioxide | Carbon dioxide | Pure PO | OH number | [mPa * s/ |
| [ppm] | [g] | [g] | [mg KOH/g] | 25° C.] |
| 5000 | 0.973 | 193.6 | 58.0 | 420 |
| 1500 | 0.292 | 194.3 | 46.9 | 620 |
| 500 | 0.097 | 194.5 | 43.7 | 640 |
| 250 | 0.049 | 194.6 | 42.9 | 650 |
| 0 | 0.000 | 194.6 | 42.1 | 660 |

In the experiment using 5000 ppm of carbon dioxide, propylene carbonate could be detected in the polyol by gas chromatography.

The maximum permissible amount was determined as described in B). This was found to be 500 ppm.

The experiments thus demonstrate the advantages of the process of the invention over the conventional processes.

The above-described problems encountered in conventional processes, i.e. using alkylene oxides which are contaminated to a greater extent with interfering substances as starting materials, are avoided in the process of the invention. For example, there is no significant discoloration due to aldol condensations caused by an excessively high content of aldehydes or undesirable and unforeseeable deviations in the properties of the polyols, and thus the polyurethanes which can be produced therefrom, due to the influence of excessively high contents of allyl alcohol in the alkylene oxides used.

1) A process for preparing polyetherols in the presence of basic catalysts, wherein propylene oxide (PO) and/or ethylene oxide (EO) whose summated contents
1a) of aldehydes, expressed by the contents determined by gas chromatography or titrimetrically using the bisulfite method and calculated for propionaldehyde, do not exceed the value of 300 ppm and
1b) of allyl alcohol determined by gas chromatography do not exceed the value of 2500 ppm and
1c) of water determined by Karl-Fischer titration do not exceed the value of 1700 ppm and
1d) of acid, expressed by the contents determined by acid-base titration and calculated for acetic acid, do not exceed the value of 100 ppm and
1e) of carbon dioxide determined by KOH titration do not exceed the value of 500 ppm
are used.

2) The process for the base-catalyzed preparation of polyetherols according to claim 1, wherein a propylene oxide (PO) and/or an ethylene oxide (EO) whose summated contents of aldehydes, expressed by the contents determined by gas chromatography or titrimetrically using the bisulfite method and calculated for propionaldehyde, do not exceed the value of 120 ppm, are used.

3) The process for the base-catalyzed preparation of polyetherols according to claim 1 or 2, wherein a propylene oxide (PO) and/or an ethylene oxide (EO) whose summated contents of allyl alcohol determined by gas chromatography do not exceed the value of 1000 ppm are used.

4) The process for the base-catalyzed preparation of polyetherols according to any of claims 1-3, wherein a propylene oxide (PO) and/or an ethylene oxide (EO) whose summated contents of water determined by Karl-Fischer titration do not exceed the value of 500 ppm, preferably 200 ppm, are used.

5) The process for the base-catalyzed preparation of polyetherols according to any of claims 1-4, wherein a propylene oxide (PO) and/or an ethylene oxide (EO) whose summated contents of acid, expressed by the contents determined by acid-base titration and calculated for acetic acid, do not exceed the value of 40 ppm are used.

6) The process for the base-catalyzed preparation of polyetherols according to any of claims 1-5, wherein a propylene oxide (PO) and/or an ethylene oxide (EO) whose summated contents of carbon dioxide determined by KOH titration do not exceed the value of 200 ppm are used.

7) The process according to any of claims 1-6, wherein the basic catalyst is selected from the group consisting of alkali metal hydroxides and alkaline earth metal hydroxides, preferably potassium hydroxide.

8) The process according to any of claims 1-7, wherein only EO is used and the content of water determined by Karl-Fischer titration does not exceed the value of 70 ppm, preferably 50 ppm.

9) A polyetherol which can be prepared by the process according to any of claims 1-8.

10) The use of a polyetherol according to claim 9 for producing polyurethanes.
